(19) United States
(12) Patent Application Publication
(10) Pub. No.: US 2008/0021437 A1
(43) Pub. Date: Jan. 24, 2008

(54) Enhancement of the efficacy of a clenching reduction device by augmenting with botulinum toxin type A

(76) Inventor: James P. Boyd, Rancho Santa Fe, CA (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 11/796,625

(22) Filed: Apr. 27, 2007

Related U.S. Application Data
(60) Provisional application No. 60/795,384, filed on Apr. 27, 2006.

(51) Int. Cl. A61M 31/00

(52) U.S. Cl. 604/514

(57) ABSTRACT

A method for reducing or preventing the occurrence of chronic tension headaches, migraine headaches, or temporormandibular disorders, including the application of a therapeutic, non-disabling, non-disfiguring dose of Botulinum Toxin Type A to the temporalis and/or messeter muscles of a patient, in combination with the use of a semi-custom intraoral discluder device that can be placed on either the upper teeth or the lower teeth to prevent contact of opposing upper and lower teeth. The combination of the use of an semi-custom intraoral discluder device with the administration of Botulinum Toxin Type A to the temporalis and/or messeter muscles, provides a greater reduction in maximum clenching than the use of Botulinum Toxin Type A or the semi-custom discluder device alone.

### CROSS-REFERENCE TO RELATED APPLICATION

Priority is claimed under 35 U.S.C. §119(e) to U.S. Provisional Patent Application No. 60/795,384, filed on Apr. 27, 2006, entitled “Enhancement of the efficacy of a clenching reduction device by augmenting with Botulinum Toxin Type A,” by James P. Boyd, which application is incorporated by reference herein.

### BACKGROUND OF THE INVENTION

### Field of the Invention

The present invention relates generally to intraoral devices and their methods of use and, more particularly, to a method of using an semi-custom intraoral discluder device in combination with the application of Botulinum Toxin Type A to the clenching muscles in order to reduce the intensity of nocturnal clenching and relieve tension headaches, common migraine headaches, and temporomandibular disorders.

Tension and muscle contraction headaches affect many people every day. The headaches are often recurring and, without effective treatment, can become very painful, restricting an individual's ability to think clearly and function effectively. The discomfort associated with tension and muscle contraction headaches is usually due to pain from strained and fatigued muscles of the head. The majority of the muscles of the human head are not sufficiently strong to elicit the type of pain and discomfort associated with tension and muscle contraction headaches. That is not the case with the temporalis muscle, however, which is located on the side of the skull and extends from just behind the eye to just behind the ear, and which is an extremely powerful muscle that functions to close or elevate the jaw.

Under normal circumstances, the temporalis muscle should not exert a large static force by contracting isometrically, except possibly during normal chewing. Inappropriate isometric contraction of temporalis muscle is commonly known as “clenching” and clinically known as myofacial dysfunction. Unfortunately, myofacial dysfunction is particularly difficult to detect or diagnose because the act of clenching is a relatively motionless act that is commonly done while a person is concentrating on another topic, or while sleeping.

As the muscular contraction condition of “clenching” continues, the muscle becomes fatigued and susceptible to spasm and cramping. The pain from spasm and cramping of the temporalis fibers is severe and usually diagnosed as a common migraine. Individuals suffering from headaches, who seek the assistance of a physician, are usually treated with muscle relaxants, analgesics, and physical therapy for the muscle fatigue. However, medications and therapy require continual treatment and treat only the symptoms of the underlying problem and not the problem itself.

Persons suffering from headaches, who seek the assistance of a dentist, commonly will be diagnosed with a temporomandibular disorder and treated with an intraoral “jaw positioning” appliance. Unfortunately, the intraoral appliances provided by dentists usually are not entirely effective because they only approximate the relative positions of the upper and lower teeth with respect to each other, allowing clenching to continue with minimal mandibular movement. Further, these intraoral appliances ordinarily cannot be used with patients who have malocclusions, protrusions or retrusions of the mandible, or other irregular teeth or mandibular orientations. Typically, the intraoral appliance must also be fabricated by a dentist at a prohibitive cost to a majority of individuals who suffer from tension and common migraine headaches. Lastly, most intraoral jaw positioning appliances and other types of semi-custom intraoral discluder devices can only be used on the upper teeth. However, in some circumstances, use of the device on the upper teeth is impossible due to malocclusions and irregular orientation of the teeth.

U.S. Pat. Nos. 5,513,656, 5,795,150, and 6,666,212 (hereby incorporated by reference) describe semi-custom intraoral discluder devices for preventing chronic tension headaches, common migraine headaches and temporomandibular disorders that are caused or perpetuated by chronic activity of the temporalis muscle. The discluder devices can be used with various teeth and jaw orientations and can be placed on either the upper teeth or the lower teeth to prevent contact of opposing upper and lower teeth. By preventing contact of the upper and lower teeth, the semi-custom intraoral discluder devices inhibit inappropriate isometric contraction of the temporalis muscle.

While the aforementioned devices have been shown to effectively reduce nocturnal clenching intensity, the reduction is insufficient to alleviate the symptoms caused by clenching in some patients. Therefore, there is need for a method to further reduce the intensity of nocturnal clenching. The present invention satisfies this need.

One therapeutic modality for certain neuromuscular disorders which has begun to gain acceptance in recent years is the administration of invertebrate exotoxins in a pharmaceutically safe form. For example, serotype A of the Botulinum toxin has been recommended in the art for use for the treatment of certain diseases such as disorders of the extraocular muscles (e.g., comitant strabismus and nystagmus) as well as dystonias (involuntary contractions of facial muscle) (see, e.g., The New England Journal of Medicine, 324:1186-1194, 1991). The advantage of using Botulinum Toxin Type A in this context is that it produces a reversible, flaccid paralysis of mammalian skeletal muscle, presumably by blocking the exocytosis of acetylcholine at peripheral, presynaptic cholinergic receptors, with limited activity at receptors in the central nervous system (Rabasseda, et al., Toxicon, 26:329-326, 1988). Additionally, Botulinum Toxin Type A is not believed to result in degeneration of nervous or muscular tissue and has been approved for use in certain therapies by the U.S. Food and Drug Administration.

A commercially available botulinum toxin containing pharmaceutical composition is sold under the trademark BOTOX® (available from Allergan, Inc., of Irvine, Calif.). BOTOX® consists of a purified Botulinum Toxin Type A complex, albumin and sodium chloride packaged in sterile, vacuum-dried form. The Botulinum Toxin Type A is made from a culture of the Hall strain of Clostridium botulinum grown in a medium containing N-Z amine and yeast extract. The Botulinum Toxin Type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying.

It has also been shown (U.S. Pat. No. 5,714,468) that the administration of a therapeutically effective amount of a pharmaceutically safe invertebrate presynaptic neurotoxin, such as Botulinum Toxin Type A, to a mammal can reduce migraine headaches in mammals. The Botulinum Toxin Type A is administered as an extramuscular injection, such as into the perimuscular areas of the face, cranium and neck, as well as into localized sites of pain in these areas.

Although the administration of Botulinum Toxin Type A has been shown to reduce migraines, the administration of Botulinum Toxin Type A alone in amounts necessary to therapeutically reduce migraines or other headaches renders the patient disfigured and dysfunctional (Hourglass Deformity Following BTW Injection, Headache, 20044; 44:262-264). Additionally, it is believed that the administration of Botulinum Toxin Type A to the temporalis and/or messeter muscles does not sufficiently reduce maximum clenching. Therefore, there is need for a method to further reduce the intensity of clenching. The present invention satisfies this need.

### SUMMARY OF THE INVENTION

The above-noted problems are overcome in accordance with this invention by the co-application of a non-disabling, non-disfiguring, therapeutic dose of Botulinum Toxin Type A to the clenching muscles, in combination with use of a small semi-custom intraoral discluder device that can be used with various teeth and jaw orientations and can be placed on either the upper teeth or the lower teeth to prevent contact of opposing upper and lower teeth. The use of a semi-custom intraoral discluder device, together with the administration of Botulinum Toxin Type A, inhibits inappropriate isometric contraction of the temporalis muscle, thereby preventing tension and common migraine headaches and other temporomandibular disorders. The combination of the use of a semi-custom intraoral discluder device with the administration of Botulinum Toxin Type A to the temporalis and/or messeter muscles, provides a greater reduction in maximum clenching than the use of either Botulinum Toxin Type A or a semi-custom discluder device alone.

The semi-custom intraoral discluder device of the invention includes a prefabricated trough, having a front wall and a rear wall. Extending distally from the trough is a small dome shape that extends such that as the jaws come together, the lower (mandibular) anterior incisal teeth edges come into contact with the dome prior to the upper and lower posterior teeth coming into contact. This maintains the separation, or disclusion, of the posterior teeth and prevents clenching. The dome can be modified by the wearer so that disclusion is maintained in all mandibular excursive movements.

If necessary, the trough can be filled with an adaptable material that conforms to the shape of the incisors and assists in retaining the trough on the upper or lower incisors. In addition, means can be provided for enhancing retention of the adaptable material within the trough. Such retaining means can take the form of one or more cutouts formed in a wall of the trough, a mechanical undercut in a wall of the trough, an adhesive, and/or natural attraction of the adaptable material to the trough.

In other, more detailed features of the invention, a protrusion may extend a substantial distance anteriorly and posteriorly from the trough. The protrusion is typically dome shaped and extends such that as the lower jaw is elevated, the edge of the opposing incisor or incisors comes into contact with the contact surface of the protrusion prior to the opposing upper and lower teeth coming into contact. The contact surface of the protrusion prevents the upper and lower teeth from contacting each other, regardless of the protrusive, retrusive, or excursive position of the mandible or the teeth or any mandibular movement and suppresses isometric contraction of the temporalis muscle. The protrusion can be further modified by the practitioner or wearer to accommodate for any unusual or extreme mandibular movements or teeth orientations.

Other features, and advantages of the present invention should become apparent from the following description of the preferred embodiments, taken in conjuncti on with the accompanying drawings, which illustrate, by way of example, the principles of the invention.

### BRIEF DESCRIPTION OF THE DRAWINGS

Details of the invention, and of preferred embodiments thereof, will be further understood upon reference to the drawing, wherein:

FIG. 1 is a schematic side view elevation of the human skull with the semi-custom discluder device of this invention in use;

FIG. 2 is a perspective view of the semi-custom discluder device seen from the left-front-interior;

FIG. 3 is a perspective view of the semi-custom discluder device seen from the inferior-posterior;

FIG. 4 is a perspective view of the semi-custom discluder device seen from the left-front-inferior, with the trough filled with acrylic resin and with one extending tab in place, and another extension tab aligned for placement;

FIG. 5 is an anterior view of the semi-custom discluder device in function, secured onto the anterior maxillary incisors with the discluding dome opposing the mandibular incisors.

FIG. 6 is a perspective view of an alternative embodiment of the intraoral discluder device.

FIG. 7 is a front elevational view of the intraoral discluder device of FIG. 6, in place over the maxillary teeth, opposing the mandibular incisors.

FIG. 8 is a front elevational view of the intraoral discluder device of FIG. 6, in place over the mandibular teeth, opposing the maxillary incisors.

FIG. 9 is a side sectional view of the intraoral discluder device of FIG. 6, in place over a maxillary incisor with an adaptable material conforming to the shape of the maxillary incisor, opposing a mandibular incisor, with the mandibular incisor shown in both a protrusive and a retrusive position.

FIG. 10 is a side sectional view of the intraoral discluder device of FIG. 6, in place over a mandibular incisor with an adaptable material conforming to the shape of the mandibular incisor, opposing a maxillary incisor, with the mandible in a retruded position.

FIG. 11 is a side sectional view of the intraoral discluder device of FIG. 6, similar to the view of FIG. 6, but with the mandible in a protruded position.

FIG. 12 is a perspective view of an alternative embodiment of a semicustom intraoral discluder device in accordance with the invention, including a trough filled with an adaptive material, a protrusion, and one extending tab placed on the protrusion and another extending tab aligned for placement on the first extending tab.

FIG. 13 is a perspective view of another alternative embodiment of a semi-custom intraoral discluder device in accordance with the invention, this embodiment differing from the discluder embodiment of FIG. 6 in that the front wall of its trough includes cutouts for enhancing retention of the adaptable material.

### DETAILED DESCRIPTION OF THE PREFERRED EMBODIMENTS AND METHODS

Intraoral Discluder Device

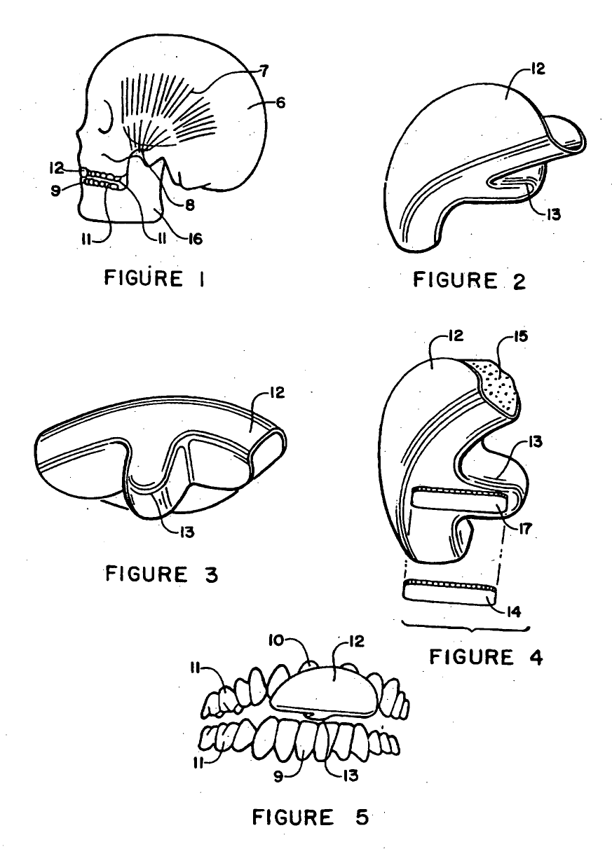

Referring now to FIG. 1, there is seen a schematic representation of a human skull 6. The temporalis muscle 7 extends from the skull to it's attachment 8 on the mandible (jaw) 16, with contraction of the muscle 7 causing the jaw 16 to close. When the discluder 12 of this invention (as detailed in FIGS. 2-4) is in place along the anterior maxillary teeth 10 in FIG. 1 and FIG. 5, only the anterior portion and perhaps the dome 13 is seen. As is apparent, the lower anterior teeth 9 contact the dome 13, preventing posterior teeth 11 from coming into contact.

The intraoral discluder device 12 is shown in detail in FIGS. 2-4, which is a curved trough, similar to the curvature of the maxillary anterior teeth 10. Extending distally from the trough, is a dome 13, which the lower anterior incisors 9 come into contact with as the mandible 16 elevates. The discluder 12 is held in place on the anterior maxillary incisors 10 by a resilient material 15, such as silicone resin, placed within the trough 12 by the wearer, and which is then placed in the mouth, over the maxillary anterior incisors 10.

In the event that the posterior teeth 11 come into contact before the lower incisors 9 contact the dome 13 while the intraoral discluder device 12 is in place, extension tabs 14 can be adhered to the dome 13 by the wearer until such time that the lower incisors 9 contact the dome-with-tab complex 17 before the posterior teeth 11 contact.

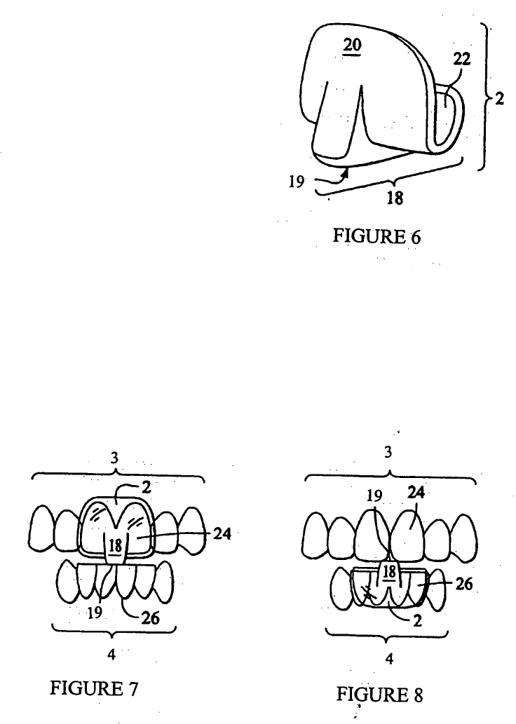

An alternative embodiment of an intraoral discluder device 2 is shown in FIG. 6. The discluder device 2 includes a trough 22 with a labial wall 20 and a protrusion 18 with an anterior contact surface 19 extending from the labial wall of the trough. The discluder device 2 may be made of any biocompatible material that will hold its form, including, e.g., polymers, enamels, rubbers, silicone resins, and any other materials that would be known to be used by those skilled in the art. In an alternative embodiment, the protrusion and the trough may be made of different biocompatible materials selected from these same examples.

FIG. 7 shows the intraoral discluder device 2 in place over the maxillary incisors 24, with the contact surface 19 of the protrusion 18 contacting the opposing mandibular incisors 26 when the mandible 16 elevates. The contact surface is positioned a sufficient distance away from the trough 22 to prevent the opposing upper teeth 3 and lower teeth 4 from contacting each other. Typically, this distance is on the order of several millimeters.

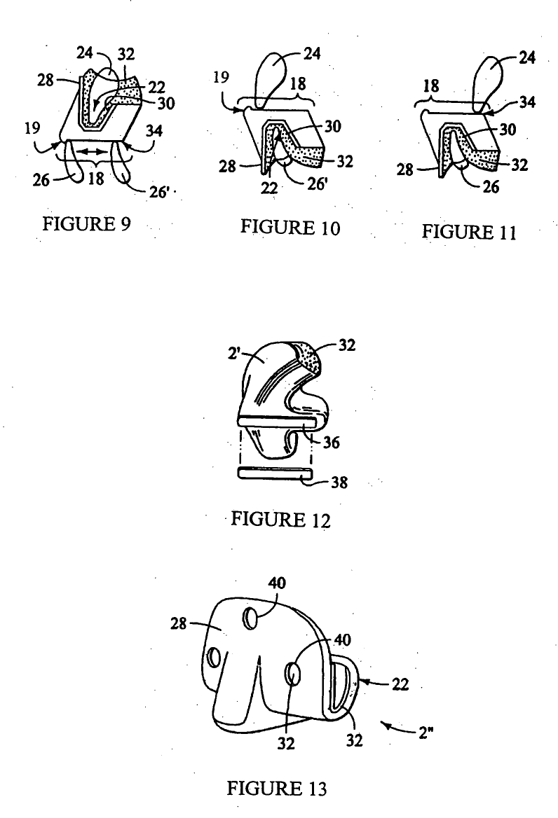

With reference now to FIG. 9, the trough 22 of the intraoral discluder device 2 is defined by a front wall 28 and a rear wall 30. An adaptive material 32 can optionally be disposed within the trough, for conforming engagement with the maxillary incisors 24. This adaptive material may be made of any type of material that conforms and retains its shape, including, e.g., silicone resins, polymers, enamels, rubbers, and any other material that would be known to be used by those skilled in the art. This material aids in providing a comfortable and durable engagement between the discluder and the incisors.

In one feature of the invention, the protrusion 18 projects both anteriorly and posteriorly from the trough 22. This ensures that the opposing mandibular incisors 26 will contact the contact surface 19 of the protrusion 18 regardless of whether the mandible is in a protrusive position or a retrusive position. These two positions are depicted in FIG. 9, with the mandibular incisor being identified by the reference numeral 26 when it is in a protrusive position and by the reference numeral 26′ when it is in a retrusive position. Preferably, the protrusion has a length in the anterior/posterior direction in the range of about 8 mm to about 12 mm. It projects anteriorly from the front wall 28 of the trough by at least about 3 mm.

Alternatively, as shown in FIG. 8, the intraoral discluder device 2 can be placed over the mandibular incisors 26, with the contact surface 19 of the protrusion 18 contacting the opposing maxillary incisors 24 when the mandible 16 elevates. As in the case when the discluder is placed over the maxillary incisors, this prevents the opposing upper and lower teeth 3, 4 from contacting each other.

FIGS. 10 and 11 show the intraoral discluder device 2 positioned on the mandibular incisors, with its trough 22 being held in place by the adaptive material 32 around the mandibular incisor 26. In FIG. 10, the opposing maxillary incisor 24 contacts an anterior portion of the contact surface 19 of the protrusion 18 when the mandible is in a retrusive position. In FIG. 11, on the other hand, the opposing maxillary incisor contacts a posterior portion of the contact surface when the mandible is in a protrusive position.

An alternative embodiment of an intraoral discluder device 2′ in accordance with the invention is depicted in FIG. 12. It includes a trough 22 and a protrusion 18 projecting both anteriorly and posteriorly from the trough. An adaptive material 32 is disposed within the trough, and two extending tabs 36, 38 are included for placement on the protrusion, to increase the distance of the contacting surface 19 from the trough. The extending tab 36 is shown secured to the protrusion, and the extending tab 38 is shown in alignment with the tab 36. These tabs are selectively used if the wearer's mouth is configured such that the upper teeth 3 and the lower teeth 4 contact each other before the opposing incisors 24 or 26 contact the protrusion. The wearer or practitioner can selectively adhere one or more of these extension tabs to the occluding face of the protrusion to increase the distance between the contact surface and the trough. The extension tabs may be made of any suitable biocompatible material, including, e.g., silicone resins, polymers, enamels, rubbers, and any other material known to those skilled in the art. The extension tabs may be adhered to the entire protrusion, as shown, or to only a portion of the protrusion. The extension tabs may be adhered by any suitable method, including, e.g., adhesives, cutouts, prefabricated snap-in-place pieces, natural attraction, adhesion, or other any other suitable method known to those skilled in the art.

Another alternative embodiment of a semi-custom intraoral discluder device 2″ in accordance with the invention is depicted in FIG. 14. This discluder includes a trough 22 and a protrusion 18 projecting both anteriorly and posteriorly from the trough, and an adaptive material 32 is disposed within the trough. This discluder further includes three circular cutouts 40 in the trough's front wall 28, for enhancing the retention of the adaptive material within the trough. Other structures for enhancing retention of the adaptive material could include mechanical undercuts, adhesives, and/or natural attraction of the adaptable material to the trough.

It should be evident from the drawings and the discussion above that the semi-custom intraoral discluder device 2 of the invention may be used on either the upper teeth 3 or lower teeth 4 and with various teeth and jaw orientations, to prevent the upper teeth and lower teeth from contacting each other and causing inappropriate isometric contractions of the temporalis muscle 6. The semi-custom intraoral discluder device of the invention prevents tension and common migraine headaches and temporomandibular disorders that result from inappropriate isometric contraction of the temporalis muscle. The use of an semi-custom intraoral discluder device has been shown to reduce to maximum clenching. However, the reduction is insufficient to alleviate the symptoms caused by isometric contraction of the temporalis muscle in some patients.

Administration of Botulinum Toxin Type A

The administration of a therapeutic, non-disabling, non-disfiguring dosage of Botulinum Toxin Type A (similar to dosages currently approved by the FDA for the treatment of glabellar lines, for example) into the temporalis and/or masseter muscles of a patient, in combination with the use of an intraoral semi-custom discluder device (as described above), provides a greater reduction in maximum clenching than the use of Botulinum Toxin Type A or a semi-custom discluder device alone, without causing disfiguration to the patient. The combination also prevents the adverse effects caused by administering Botulinum Toxin Type A alone. For example, the combination prevents the hourglass look that results from atrophy of the temporalis muscles caused by the administration of larger, therapeutic doses of Botulinum Toxin Type A alone.

The dosage of Botulinum Toxin Type A administered to a patient is dependent upon the size of the temporalis and masseter muscles—the larger the muscles, the larger the dose of Botulinum Toxin Type A. A larger dose of Botulinum Toxin Type A is also administered if the muscle is hypertrophied.

In a preferred embodiment, approximately 100 units of Botulinum Toxin Type A is diluted in 2 cc of normal saline. A dosage of between about 20 and about 40 units is administered to each temporalis muscle, and a dosage of about 10 units is administered to each masseter muscle. Do you have any further dosage information or instructions to add?

In a series of tests, it was shown that the use of an semi-custom intraoral discluder device alone resulted in a reduction of maximum clenching by approximately 65%. Whereas, the administration of Botulinum Toxin Type A alone resulted in a reduction of maximum clenching by approximately 40%. The tests also showed that the combination of the use of an semi-custom intraoral discluder device with the administration of a therapeutic, non-disabling, non-disfiguring dosage of Botulinum Toxin Type A to the temporalis and/or messeter muscles, resulted in a reduction of maximum clenching by approximately 85%.

Although the invention has been described in detail with reference to the presently preferred embodiments, those of ordinary skill in the art will appreciate that various modifications can be made without departing from the invention. Accordingly, the invention is defined only by the following claims.

What is claimed is:

1. A method for reducing or preventing the occurrence of chronic tension, migraine headaches, or temporormandibular disorders comprising the steps of:
providing a semi-custom intraoral discluder device that includes a prefabricated trough shaped to extend over at least a portion of the anterior and posterior surfaces of the anterior maxillary incisors in a spaced relationship therewith, a dome on said trough extending distally when said trough is in place adjacent to said anterior maxillary incisors, wherein said dome is configured to engage at least one lower anterior incisor prior to any contact between upper and lower posterior teeth while substantially avoiding lateral pressure on said at least one lower anterior incisor; and
administering a therapeutic, non-disabling, non-disfiguring dosage of Botulinum Toxin Type A to a temporalis muscle of a patient.

2. The method according to claim 1, and further including a step of placing a quantity of adaptable material into the trough, such material conforming to the shape of the at least one upper or lower incisor, thereby assisting in retaining the device in the wear's mouth.

3. The method according to claim 2, wherein the semi-custom intraoral discluder device further includes means for retaining the adaptable material within the trough.

4. The method according to claim 3, wherein the means for retaining is selected from the group consisting of one or more cutouts formed in a wall of the trough, a mechanical undercut in a wall of the trough, an adhesive, and natural attraction of the adaptable material to the trough.

5. The method according to claim 1, wherein the trough is fabricated from a biocompatible material.

6. The method according to claim 1, wherein said trough and dome are fabricated from a synthetic resin.

7. The method according to claim 1, wherein the semi-custom intraoral discluder device further includes a plurality of tabs fastened over said dome to increase the height thereof as desired.

8. A method for reducing or preventing the occurrence of chronic tension, migraine headaches, or temporormandibular disorders comprising the steps of:
providing a semi-custom intraoral discluder device that includes a trough defining a first axis and having a front wall and a rear wall sized and configured to accommodate at least one upper or lower incisor, wherein the front wall is disposed adjacent to the front of the incisor and the rear wall is disposed adjacent to the rear of the incisor when the trough is in the wearer's mouth, and a protrusion attached to the trough and projecting anteriorly from the front wall of the trough and a posteriorly from the rear wall of the trough when the tough is in the wearer's mouth, wherein the protrusion defines a second axis generally perpendicular to the first axis of the trough, and wherein the protrusion is sized and configured to prevent contact between opposing upper and lower teeth; and
administering a therapeutic, non-disabling, non-disfiguring dosage of Botulinum Toxin Type A to a temporalis muscle of a patient.

9. The method according to claim 8, and further including a step of placing a quantity of adaptable material into the trough, such material conforming to the shape of the at least one upper or lower incisor, thereby assisting in retaining the device in the wear's mouth.

10. The method according to claim 9, wherein the semi-custom intraoral discluder device provided further includes means for retaining the adaptable material within the trough.

11. The method according to claim 10, wherein the means for retaining is selected from the group consisting of one or more cutouts formed in a wall of the trough, a mechanical undercut in a wall of the trough, an adhesive, and natural attraction of the adaptable material to the trough.

12. The method according to claim 8, wherein the trough and protrusion are fabricated from a biocompatible material.

13. The method according to claim 8, and further including a step of securing at least one prefabricated tab to the protrusion, to increase the distance from at least one opposing incisor to the trough.

14. The method according to claim 8, wherein the protrusion is configured not to move when contacted by at least one opposing incisor.

15. The method according to claim 8, wherein the protrusion has a length in the anterior/posterior direction in the range of about 8 mm to about 12 mm.

16. The method according to claim 8, wherein the protrusion projects anteriorly from the front wall by at least about 3 mm.

17. A method for reducing or preventing the occurrence of chronic tension, migraine headaches, or temporormandibular disorders comprising the steps of:
providing a semi-custom intraoral discluder device that includes a trough defining a first axis and having a front wall and a rear wall sized and configured to accommodate at least one upper or lower incisor, wherein the front wall is disposed adjacent to the front of the incisor and the rear wall is disposed adjacent to the rear of the incisor when the trough is in the wearer's mouth, and a protrusion attached to the trough and projecting anteriorly from the front wall of the trough and a posteriorly from the rear wall of the trough when the tough is in the wearer's mouth, wherein the protrusion defines a second axis generally perpendicular to the first axis of the trough, and wherein the protrusion is sized and configured to prevent contact between opposing upper and lower teeth; and
administering a therapeutic, non-disabling, non-disfiguring dosage of Botulinum Toxin Type A to a temporalis muscle and a masseter muscle of a patient.

18. A method for reducing or preventing the occurrence of chronic tension, migraine headaches, or temporormandibular disorders comprising the steps of:
providing a semi-custom intraoral discluder device that includes a prefabricated trough shaped to extend over at least a portion of the anterior and posterior surfaces of the anterior maxillary incisors in a spaced relationship therewith, a dome on said trough extending distally when said trough is in place adjacent to said anterior maxillary incisors, wherein said dome is configured to engage at least one lower anterior incisor prior to any contact between upper and lower posterior teeth while substantially avoiding lateral pressure on said at least one lower anterior incisor; and
administering a therapeutic, non-disabling, non-disfiguring dosage of Botulinum Toxin Type A to a temporalis muscle and a masseter muscle of a patient.
